(19) United States
(12) Patent Application Publication
(10) Pub. No.: US 2014/0148606 A1
(43) Pub. Date: May 29, 2014

(54) ANTI-TUMOR EFFECTS OF ALLOSTERIC FOCAL ADHESION KINASE INHIBITOR, METHYL VIOLET DERIVATIVES

(71) Applicant: Korea Institute of Science and Technology, Seoul (KR)

(72) Inventors: Tae Bo SIM, Daegu (KR); Hwan KIM, Goyang (KR)

(73) Assignee: Korea Institute of Science and Technology, Seoul (KR)

(21) Appl. No.: 14/066,020

(22) Filed: Oct. 29, 2013

(30) Foreign Application Priority Data
Nov. 29, 2012 (KR) 10-2012-0136617

(51) Int. Cl. C07C 251/20

(52) U.S. Cl. CPC C07C 251/20
USPC 552/302

(57) ABSTRACT

Disclosed is a methyl violet derivative having inhibitory activity against allosteric focal adhesion kinase (FAK) and is useful as an agent for preventing or treating cancer.

### CROSS-REFERENCE TO RELATED APPLICATIONS

This application claims priority under 35 U.S.C. §119 to Korean Patent Application No. 10-2012-0136617, filed on Nov. 29, 2012, in the Korean Intellectual Property Office, the disclosure of which is incorporated herein by reference in its entirety.

### BACKGROUND

(a) Technical Field

Disclosed is a methyl violet derivative having inhibitory activity against allosteric focal adhesion kinase (FAK) and is useful as an agent for preventing or treating cancer.

(b) Background Art

Methyl violet is a deep purple alkaline dye used as an indicator. It appears yellow in solutions of low pH and changes to violet under basic condition. Its main use is for textiles, paint, ink, stamp ink and Gram stain. Most recently, it was included as an indicator in a moisture absorbent to change the color depending on the absorbent level [Korean Patent Application No. 1,046,774]. As such, methyl violet has been mainly used as a dye or an indicator.

Focal adhesion kinase (FAK) is a non-receptor tyrosine kinase that is recruited to integrin in response to signaling from growth factors. FAK is associated with adhesion, spreading, division, motility, invasion, angiogenesis, differentiation and survival. Most reports underline the activity of FAK in human progressive cancer cells including colorectal cancer, rectal cancer, skin cancer, breast cancer, lung cancer, brain cancer, prostate cancer, lymphoma, multiple myeloma and melanoma [Weiner et al., Lancet, 342 (8878): 1024-25 (1993); Owens et al., Cancer Res., 55: 2752-55 (1995); Maung et al., Oncogene, 18: 6824-28 (1999); Wang et al., J. Cell Sci. 113: 4221-30 (2000)].

Several small molecules capable of inhibiting the FAK have been reported. They are TAE226, PF-573228, PF-562271, PF-04554878 and GSK2256098 and are currently being investigated in clinical testing. Most of the disclosed FAK inhibitors are identified as ATP-competitive inhibitors. Recently, a few compounds that recognize the ATP non-competitive inhibitor have been reported to inhibit FAK. They are the four compounds: C4, INT2-31, Y11 and Y15. C4 disturbs the protein-protein interaction with VEGFR and FAK, and INT2-31 disrupts the complex IGFR and FAK. Y11 and Y15 possess selective inhibitory properties against the autophosphorylation site FAK Y397.

Until now, methyl violet and its derivatives are used only as a dye or an indicator, and it has never been reported that methyl violet exhibits strong inhibitory effect against FAK as an anti-cancer agent.

### SUMMARY

The present invention is directed to providing a methyl violet derivative as a prevention and treatment agent of cancer.

In an aspect, the present invention provides an anti-cancer agent including a methyl violet derivative represented by Chemical Formula 1 as an active ingredient:

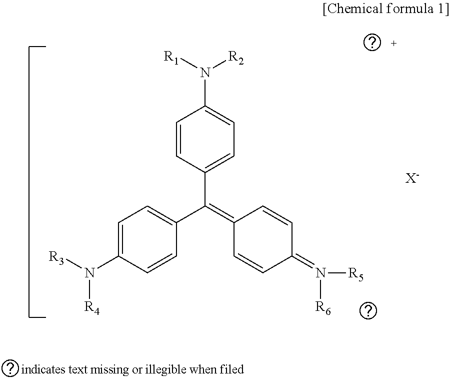

wherein each of R₁-R₆ is hydrogen or C₁-C₁₀ alkyl and wherein each of R₁-R₆ is hydrogen or C₁-C₁₀ alkyl and X⁻ is a halide ion.

Other features and aspects of the present invention will be apparent from the following detailed description, drawings and claims.

### BRIEF DESCRIPTION OF THE DRAWINGS

The above and other objects, features and advantages of the present invention will now be described in detail with reference to certain exemplary embodiments thereof illustrated in the accompanying drawings which are given hereinbelow by way of illustration only, and thus are not limitative of the invention, and wherein:

FIG. 1 shows inhibitory activity of methyl violet 2B against FAK;

FIG. 2 shows non-ATP competitive inhibition of methyl violet 2B;

FIG. 3 shows inhibitory activity of methyl violet 2B against FAK signaling in cancer cell;

FIG. 4 shows signaling pathway regulation of methyl violet 2B in cancer cell;

FIG. 5 shows inhibitory activity of methyl violet 2B against invasion in cancer cell; and

FIG. 6 shows inhibitory activity of methyl violet 2B against migration in cancer cell.

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The present invention provides a novel use of a methyl violet derivative represented by the Chemical Formula 1, which is mainly used as a dye or an indicator, as an agent for preventing or treating cancer.

That is to say, the present invention provides an anti-cancer agent including a methyl violet derivative represented by Chemical Formula 1 as an active ingredient:

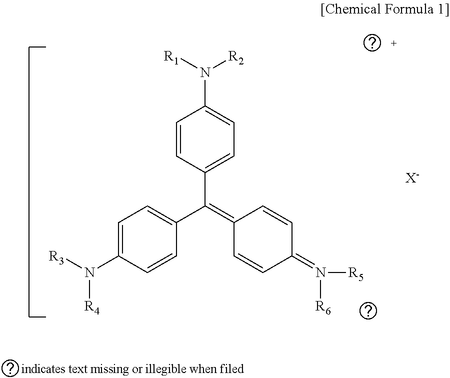

wherein each of R₁-R₆ is hydrogen or C₁-C₁₀ alkyl and wherein each of R₁-R₆ is hydrogen or C₁-C₁₀ alkyl and X⁻ is a halide ion.

Specifically, the anti-cancer agent includes a compound represented by Chemical Formula 1 wherein each of R₁-R₅ is C₁-C₆ alkyl, R₆ is hydrogen or C₁-C₆ alkyl and X⁻ is a halide ion as an active ingredient.

More specifically, the present invention provides an anti-cancer agent including methyl violet 2B represented by Chemical Formula la, methyl violet 10B represented by Chemical Formula 1b or a mixture thereof as an active ingredient:

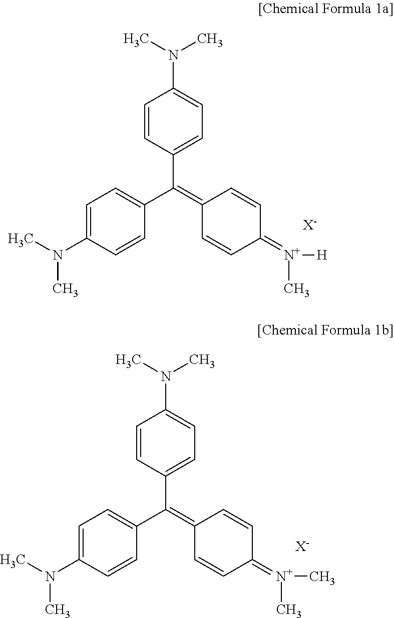

wherein X⁻ is a halide ion.

According to the experiments conducted by the inventors of the present invention, the methyl violet derivatives represented by Chemical Formula 1 have excellent inhibitory effects against FAK. Accordingly, they can be used as active ingredients in pharmaceutical compositions for preventing and treating various cancers.

The cancers that can be prevented or treated with the anti-cancer agent of the present invention may include colorectal cancer, rectal cancer, skin cancer, breast cancer, lung cancer, brain cancer, prostate cancer, lymphoma, multiple myeloma, melanoma, or the like.

The anti-cancer agent of the present invention includes the methyl violet derivative represented by Chemical Formula 1 as an active ingredient and may further include a commonly used, non-toxic, pharmaceutically acceptable carrier, adjuvant, excipient, or the like to prepare formulations for oral administration such as tablet, capsule, troche, liquid, suspension, etc. and formulations for parenteral administration.

The excipient that may be used in the anti-cancer agent of the present invention includes a sweetener, a binder, a solubilizer, a wetting agent, an emulsifier, an isotonic agent, an adsorbent, a disintegrant, an antioxidant, a preservative, a lubricant, a filler, an aromatic, or the like. For example, lactose, dextrose, sucrose, mannitol, sorbitol, cellulose, glycine, silica, talc, stearic acid, stearin, magnesium stearate, magnesium aluminum silicate, starch, gelatin, gum tragacanth, alginic acid, sodium alginate, methyl cellulose, sodium carboxymethyl cellulose, agar, water, ethanol, polyethylene glycol, polyvinylpyrrolidone, sodium chloride, calcium chloride, orange essence, strawberry essence, vanilla flavor, or the like may be used.

The administration dose of the anti-cancer agent according to the present invention may vary depending on the patient's age, body weight, sex and physical conditions, administration type, severity of disease, etc. Based on an adult patient weighing 70 kg, the administration dose may be in general 0.01 to 1000 mg/day. As per the decision by a physician or a pharmacist, the administration may be made once or several times a day with predetermined time intervals.

### EXAMPLES

The present invention will be described in more detail through examples. The following examples are for illustrative purposes only and it will be apparent to those skilled in the art not that the scope of this invention is not limited by the examples.

### Example 1

### Cell Culture & Cancer-Specific Anti-Proliferative Activity Assay

Melanoma cells (A375P, A375SM) and breast cancer cells (MDA-MB-231, MCF7) were maintained in DMEM medium (Welgene, Daegu, Korea). Colorectal cancer cells (HCT116, HT29), lung cancer cells (A549) and prostate cancer cells (DU145) were grown in RPMI1640 medium (Welgene). NIH3T3 and Balb/c cells were cultured in DMEM/F12 medium (Welgene).

Each medium was supplemented with 10% FBS (Welgene) and 1% penicillin/streptomycin (Welgene) in a humidified atmosphere with 5% CO₂ at 37° C. A375P cells were purchased from the American Type Culture Collection (ATCC, Rockville, Md., US), and A375SM, MDA-MB-231, MCF7, HCT116, HT29, A549, DU145, NIH3T3 and Balb/c were purchased from the Korean Cell Line Bank (Seoul, Korea).

Cells were taken from culture substrate with 0.05% trypsin-0.02% EDTA and plated at a density of 5×10³ cells/well in 96 well plates and then incubated at 37° C. for 24 h in a humidified atmosphere with 5% CO₂ prior to treatment of various concentration (tenfold serial dilution, 6 steps) of test compounds. The cell viability was assessed by the conventional 3-(4,5-dimethylthiazol-2-yl)-2,5-diphenyltetrazolium bromide (MTT) reduction assay and luminescent assay. The MTT and luminescent assays were carried out with CellTiter 96® and CellTiter-Glo® Luminescent Cell Viability Assay (Promega, Wis., USA) according to the manufacturer's instructions. The absorbance at 590 nm was recorded using EnVision2103 (PerkinElmer; Boston, Mass., US). The IC₅₀ value was calculated using the GraphPad Prism 4.0 software.

The inhibitory activity against proliferation of the various cancer cell of methyl violet 2B according to the present invention is given in Table 1.

TABLE 1

| Cancer cell | Organ | IC₅₀ (nM) |
|---|---|---|
| A375P | skin | 19.58 |
| A375SM | skin | 34.12 |
| MDA-MB-231 | breast | 13.39 |
| MCF7 | breast | 69.73 |
| HCT116 | colon | 36.86 |
| HT29 | colon | 18.07 |
| A549 | lung | 45.56 |
| DU145 | prostate | 81.45 |
| NIH3T3 | fibroblast | 938.1 |
| Balb/c | fibroblast | 59370 |

As listed in Table 1, the compound of the present invention exhibited stronger antiproliferative activities (double-digit nM of IC₅₀) in various cancer cells. In addition, the inhibition of proliferation of normal fibroblasts (NIH3T3 and Balb/c) was 10 to 1,000-fold less (GI₅₀=1-10 μM) compared to that of the cancer cells. The MTT assay (absorbance reading) data were compared with viability data derived from the CellTiter-Glo® assay, a luminescent cell viability assay. No significant difference was observed between the two sets of cell viability data.

In the context of the experimental results, anti-proliferative activity measurement against several different cancer cells showed that the compound of the present invention is capable of significantly suppressing the proliferation of cancer cells but not normal fibroblasts.

### Example 2

### FAK Biochemical Kinase Assay

Kinase reactions were performed at room temperature in lysis buffer (50 mM HEPES pH 7.5, 1 mM EGTA, 10 mM MgCl₂, 2 mM DTT and 0.01% Tween-20) with 0.3 nM FAK (Signalchem), 100 nM ULight-poly GT (PerkinElmer), 10 μM ATP, and various concentrations (10 fold serial dilution, 6 points) of test compounds at a final volume of 10 μL. After 1 h, the kinase reaction was stopped by adding 10 mM EDTA and progressed by incubating the reaction mixture at room temperature for 5 min. To the mixture was added Detection Mix (Eu-anti-phospho-Tyr (PT66)) antibody to a final concentration of 2 nM and incubated for 1 h at room temperature. The plates were covered before being read using an EnVision® Multilabel reader in TR-FRET mode (excitation at 320 nm and emission at 665 nm, PerkinElmer).

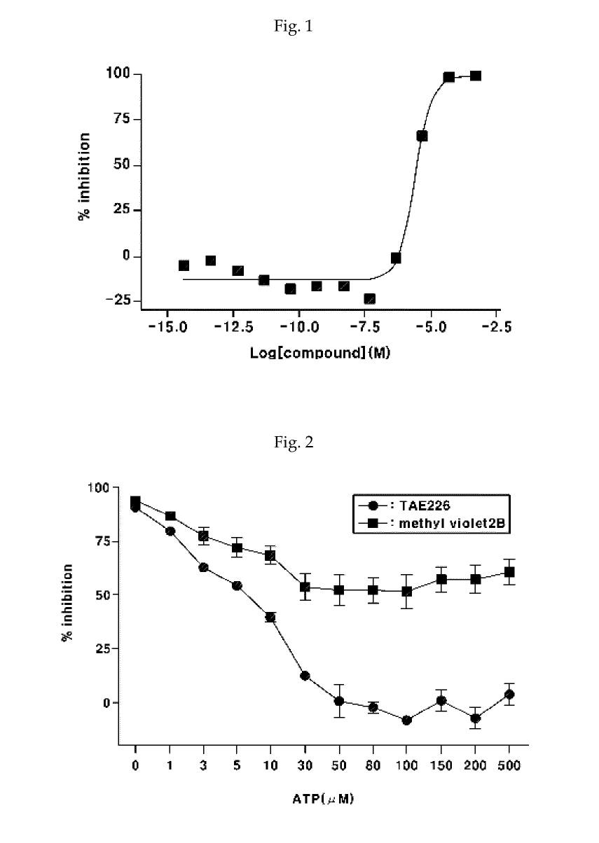

FIG. 1 indicates that methyl violet 2B moderately inhibits the kinase activity of full-length FAK in biochemical assays. Full-length FAK biochemical kinase assays were carried out to estimate the IC₅₀ value of methyl violet 2B. Methyl violet 2B was found to have an IC₅₀ of 2.7 μM (FIG. 1) in the fluorescence LANCE® Ultra kinase assay system.

FIG. 2 revealed that methyl violet 2B is a non-ATP competitive (allosteric kinase) FAK inhibitor. TAE226 was used as a reference compound, which is a known ATP competitive inhibitor of FAK. The kinase inhibitory activity of methyl violet 2B (2.7 μM) against full-length FAK was found to be independent of the concentration of ATP whereas the kinase inhibitory activity of TAE226 was inversely proportional to the concentration of ATP (FIG. 2). Thus, it was confirmed that methyl violet 2B is a non-ATP competitive allosteric inhibitor, which binds to a site remote from the ATP-binding site of FAK.

### Example 3

### Western Analysis

After incubating with the test compound for 48 h, both adherent and floating cancer cells were collected. The cells were lysed on ice in lysis buffer (50 mM Tris, pH 7.4, 150 mM NaCl, 1 mM EDTA, 1% NP-40, 0.25% sodium deoxycholate) supplemented with phosphatase inhibitors (Sigma #P2850, #5726; Phosphatase Inhibitor Cocktail I & II) and protease inhibitors (Complete, Mini, EDTA-free; Roche Applied Science) for 20 min. The protein concentration was determined using the Bio-Rad Protein assay reagent (Bio-Rad #500-0006). The lysate was centrifuged at 13000 rpm at 4° C. for 20 min. 50 μg of total proteins were mixed with 50 μg loading buffer (50 mM Tris-HCl (pH 6.8), 2% SDS, 10% 2-mercaptoethanol, 10% glycerol, and 0.002% bromophenol blue), boiled for 5 min, and subjected to a 10% SDS polyacrylamide gel electrophoresis. The proteins were electrophoresed onto nitrocellulose membranes and detected with antibodies against FER, FAK, DOCK180, Pyk2, Paxillin, AKT, GSK3, PTEN, JNK, ERK, Bcl-2, C-raf and Actin and against phosphorylated FER Tyr402, FAK Tyr925, Tyr861, Tyr577, Tyr576, Tyr397, Pyk2 Tyr402, Paxillin Tyr118, AKT S473, T308, GSK3β S9, JNK, ERK, Bad, C-raf and ERM T567, T564, T558 followed by the addition of horseradish peroxidase (HRP)-conjugated secondary antibodies and 3,3-diaminobenzidine tetrahydrochloride (DAB) as the HRP substrate.

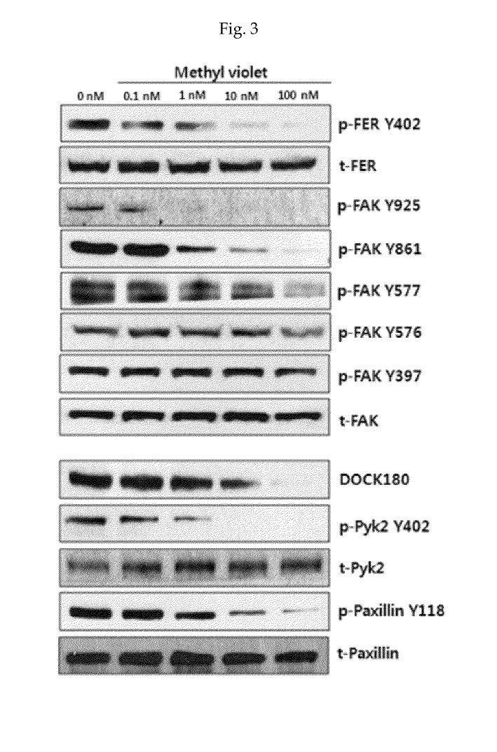

FIG. 3 shows the effect of methyl violet 2B on the intracellular FAK signaling pathway of the A375P human melanoma cell line. It was confirmed that the inhibitory effect of methyl violet 2B (0.1-10 nM) on phosphorylation of Tyr925, Tyr861 and Tyr577 in A375P cells is much greater than that of Y576 and Y397. It was also observed that methyl violet 2B significantly inhibits the phosphorylation of FER Tyr402 in A375P even at very low concentration (10 nM).

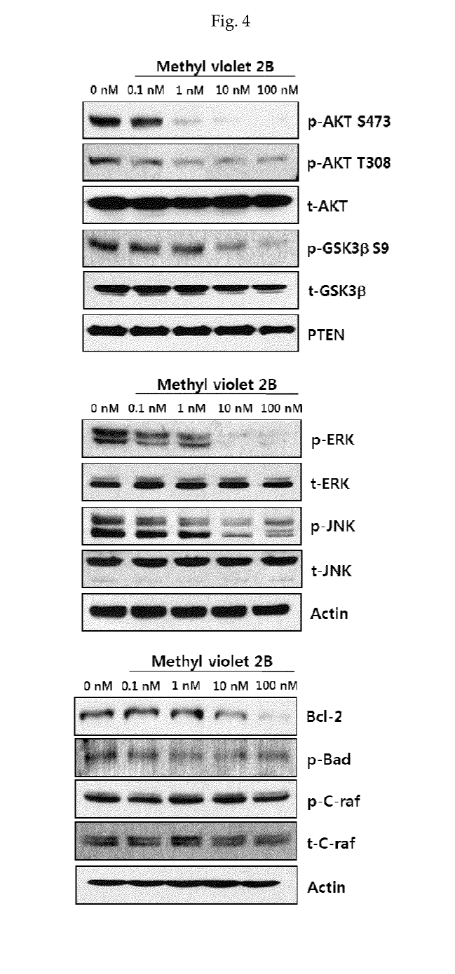

The effect of methyl violet 2B on AKT phosphorylation, known as downstream signaling of FAK, in A375P cells is shown in FIG. 4. It was found out that this substance dramatically inhibits p-AKT Ser473 and p-AKT Thr308 even at a concentration of 1 nM (FIG. 4). In addition, the phosphorylation of GSK3β S9, the AKT downstream substrate, is significantly blocked by methyl violet 2B in a dose-dependent manner but the level of PTEN is not altered by this substance. Thus, it can be seen that the inhibition of the AKT signaling pathway was caused by the blocking of FAK activity by methyl violet.

Also, the result of investigating the effect on MAPK signaling, known as downstream signaling of FAK, in A375P cells is shown in FIG. 4. Methyl violet 2B displays a strong inhibitory effect against p-ERK and p-JNK even at 10 nM (FIG. 4). Thus, it was confirmed that the inhibition of the MAPK signaling pathway was caused by the blocking of FAK activity by methyl violet in the A375P cells.

FIG. 4 also shows the result of testing the apoptotic effect of methyl violet 2B. At concentrations >1 nM, methyl violet significantly suppressed the expression of the anti-apoptotic molecule Bcl-2 in a concentration-dependent manner (FIG. 4). Methyl violet was less effective on p-Bad as compared to Bcl-2. Methyl violet did not affect the phosphorylation of C-raf known to be a Bcl-2 regulator. Overall, methyl violet disrupted the signaling pathway including FAK/AKT/MAPK, which is related to the cell survival and proliferation, caused the inhibition of tumor cell growth and proliferation, and induced programed cell death through the inhibition of Bcl-2 apoptotic molecule.

### Example 4

### Invasion & Migration Assay

Invasion of A375P cells was assessed by using the QCM ECMatrix Cell Invasion Assay (Chemicon, Millipore, Calif.) according to the manufacturer's protocol. The cells were plated on the membrane of the upper chamber of the transwell at a concentration of 2.5×10⁵ cells/well in 500 μL of serum-free medium. The medium in the lower chamber contained the test compound and 10% fetal bovine serum. The cells that passed through the polycarbonate membrane were stained with cell stain solution supplied in the Transwell Invasion assay (Chemicon, Millipore, Calif.) and photographed after 48 h of incubation. The invasion was quantitated by dissolving the stained cells in 10% acetic acid and transferring a consistent amount of the dye/solute mixture to a 96-well plate for colorimetric reading of OD at 560 nm.

Migration of the A375P cells was assessed by using the Culture-Inserts (Ibidi) according to the manufacturer's protocol. The cells were plated at a density of 1×10⁵ cells/well in each well of Culture-Inserts. After 24 h incubation, the Culture-Inserts were removed, and wounds were created by using a 500-μm thick wall. The dish containing these cells was treated with medium containing the compound and incubated at 37° C. for 48 h. Phase-contrast images of the closed gap wound were captured using a microscope.

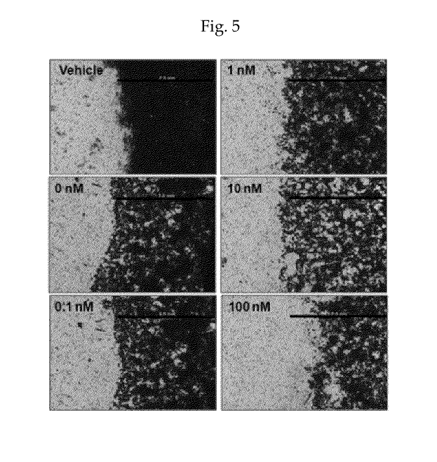

FIG. 5 shows the effect of methyl violet 2B on the invasion of cancer cells in the A375P melanoma cell line. The results of a QCM ECMatrix Cell Invasion Assay reveal that this substance (1 nM) causes a dose-dependent decrease in invasion of the A375P cells (FIG. 5).

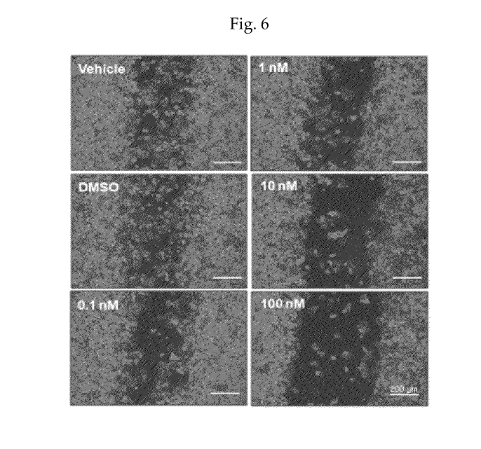

FIG. 6 reveals that methyl violet 2B potently inhibits migration. Also, the results of a wound healing assay using the Ibidi Culture-Insert show that methyl violet 2B suppresses cancer cell (A375P) motility at 1 nM concentration (FIG. 6).

Taken together, these results show that methyl violet 2B decreases the carcinogenic conversion to malignancy and strongly suppresses invasion/migration of tumor cells.

### Example 5

### Statistical Analysis

Statistical values are described as mean±SE of at least three independent experiments. Statistical significance was determined using the GraphPad Prism 4.0 software's t-test, and p<0.05 was considered to be statistically significant.

Since the methyl violet derivatives represented by Chemical Formula 1 have excellent inhibitory effects against FAK, they can be used as active ingredients in pharmaceutical compositions for preventing and treating various cancers.

The cancers that can be prevented or treated with the anti-cancer agent of the present invention may include colorectal cancer, rectal cancer, skin cancer, breast cancer, lung cancer, brain cancer, prostate cancer, lymphoma, multiple myeloma, melanoma, or the like.

The present invention has been described in detail with reference to specific embodiments thereof. However, it will be appreciated by those skilled in the art that various changes and modifications may be made in these embodiments without departing from the principles and spirit of the invention, the scope of which is defined in the appended claims and their equivalents.

1. An anti-cancer agent comprising a methyl violet derivative represented by Chemical Formula 1 as an active ingredient: 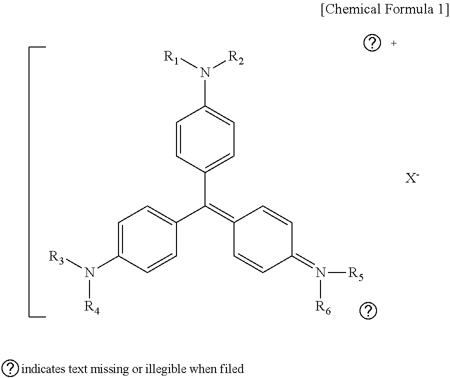
wherein each of R₁-R₆ is hydrogen or C₁-C₁₀ alkyl and X⁻ is a halide ion.

2. The anti-cancer agent according to claim 1, wherein each of R₁-R₅ is C₁-C₆ alkyl, R₆ is hydrogen or C₁-C₆ alkyl and X⁻is a halide ion.

3. The anti-cancer agent according to claim 1, wherein the methyl violet derivative is methyl violet 2B represented by Chemical Formula 1a: 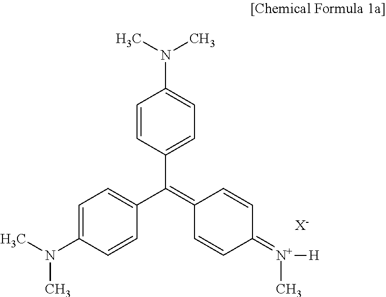
wherein X⁻ is a halide ion.

4. The anti-cancer agent according to claim 1, wherein the methyl violet derivative is methyl violet 10B represented by Chemical Formula 1b: 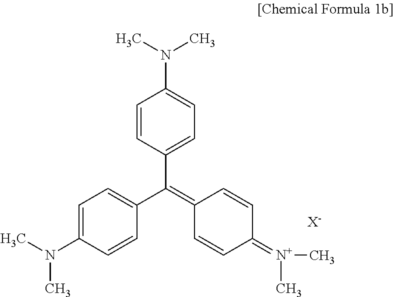
wherein X⁻ is a halide ion.

5. The anti-cancer agent according to claim 1, which exhibits inhibitory activity against focal adhesion kinase (FAK).

6. The anti-cancer agent according to claim 1, which is used to prevent and treat cancers selected from the group consisting of colorectal cancer, rectal cancer, skin cancer, breast cancer, lung cancer, brain cancer, prostate cancer, lymphoma, multiple myeloma and melanoma.

7. The anti-cancer agent according to claim 2, which exhibits inhibitory activity against focal adhesion kinase (FAK).

8. The anti-cancer agent according to claim 3, which exhibits inhibitory activity against focal adhesion kinase (FAK).

9. The anti-cancer agent according to claim 4, which exhibits inhibitory activity against focal adhesion kinase (FAK).

10. The anti-cancer agent according to claim 2, which is used to prevent and treat cancers selected from the group consisting of colorectal cancer, rectal cancer, skin cancer, breast cancer, lung cancer, brain cancer, prostate cancer, lymphoma, multiple myeloma and melanoma.

11. The anti-cancer agent according to claim 3, which is used to prevent and treat cancers selected from the group consisting of colorectal cancer, rectal cancer, skin cancer, breast cancer, lung cancer, brain cancer, prostate cancer, lymphoma, multiple myeloma and melanoma.

12. The anti-cancer agent according to claim 4, which is used to prevent and treat cancers selected from the group consisting of colorectal cancer, rectal cancer, skin cancer, breast cancer, lung cancer, brain cancer, prostate cancer, lymphoma, multiple myeloma and melanoma.
